(19) United States
(12) Patent Application Publication
(10) Pub. No.: US 2020/0324068 A1
(43) Pub. Date: Oct. 15, 2020

(54) TRACHEOSTOMY PROCEDURE KITS AND GUIDING CATHETERS

(71) Applicant: SMITHS MEDICAL INTERNATIONAL LIMITED, Ashford (GB)

(72) Inventor: Steve Chambers, Hythe (GB)

(73) Assignee: SMITHS MEDICAL INTERNATIONAL LIMITED, Ashford (GB)

(21) Appl. No.: 16/096,500

(22) PCT Filed: Mar. 30, 2017
(86) PCT No.: PCT/GB2017/000050

§ 371 Date: Oct. 25, 2018

(30) Foreign Application Priority Data
Apr. 30, 2016 (GB) 1607675.4

(51) Int. Cl. A61M 16/04; A61M 29/00

(52) U.S. Cl. CPC A61M 16/0472; A61M 2210/1032; A61M 29/00

(57) ABSTRACT

A guiding catheter (16) for use with a dilator (17) in a tracheostomy procedure has an elongated stop ring (160) the length of which is at least twice the minimum diameter D of the ring. The patient end (162) of the stop ring (160) makes a smooth transition with the external surface of the guiding catheter (16) and has the same external diameter as that of the catheter. The stop ring (160) tapers along its length so that the diameter at its machine end (163) is greater than at its patient end (162) and is the same as that of the patient end (170) of the dilator (17). In this way, the stop ring (160) makes a smooth transition with the patient end of the dilator (17).

This invention relates to guiding catheters for use with a dilator in a tracheostomy procedure.

Tracheostomy tubes are used to enable ventilation or respiration of a patient. The tube is inserted into the trachea via a surgically-formed opening in the neck so that one end locates in the trachea and the other end locates outside the patient adjacent the neck surface. Various types of different tracheostomy tubes are presently available to suit different needs. Tracheostomy tubes are also available with an inner cannula, which can be removed and replaced periodically to prevent the build-up of secretions and avoid the need to replace the tube itself. Tracheostomy tubes can be inserted by different techniques, such as the surgical cut-down procedure carried out in an operating theatre or a percutaneous dilatation procedure, which may be carried out in emergency situations.

The percutaneous tracheostomy procedure involves a number of steps in order to prepare the stoma adequately to allow a tracheostomy tube to be inserted successfully. Currently the procedure involves:

1. A needle and cannula assembly is used to puncture the tracheal wall. After the correct position has been verified, the needle is removed, leaving the cannula in position.
2. One end of a flexible guidewire is inserted into the trachea through the cannula, after which the cannula is removed, leaving the guidewire in place. A bronchoscope inserted orally may be used to monitor the procedure and avoid damage to the posterior tracheal wall.
3. One or two small (typically 14 French) pre-dilators are slid along the guidewire and through the passage into the trachea to perform an initial dilation of the tracheal wall. The pre-dilator is then removed.
4. A smaller (typically 8 French) guiding catheter is then introduced over the guide wire so that one end extends into the trachea and the other end extends externally.
5. A single stage dilator is then slid along the guiding catheter and guidewire from its machine end up to a short stop ring moulded on the outside of the guiding catheter. The dilator and guiding catheter are then advanced together into the trachea through the tracheal wall to perform the dilation. Typically the dilator is arranged to dilate the tracheal tissue to a diameter slightly larger than that of the tracheostomy tube to make placement of the tube easier. Instead of a single stage dilator, a series of dilators of increasing diameters could be used.
6. The dilator and guiding catheter are then removed, leaving the guidewire in position. The tracheostomy tube is then inserted to the trachea using a tracheostomy tube introducer.
7. Once the tracheostomy tube has been inserted correctly, the introducer and guidewire are removed through the tracheostomy tube. An inner cannula can then be inserted in the tube and the tube can be connected to a ventilator.

The numerous steps involved in this procedure bring with them various problems. For most of the time taken to complete the procedure the patient is disconnected from the ventilator so is not breathing for perhaps seven minutes or longer. The large number of components and the multiple stages involved in the procedure increase the risk of errors being made. There is also an increased risk that an item be rendered accidentally non-sterile, preventing it being used. This could mean that the patient has to be intubated with an endotracheal tube.

It is an object of the present invention to provide an alternative tracheostomy kit and guiding catheter.

According to one aspect of the present invention there is provided a guiding catheter of the above-specified kind, characterised in that the guiding catheter has an elongated stop ring the length of which is at least twice the minimum diameter of the ring, that the patient end of the stop ring makes a smooth transition with the external surface of the guiding catheter and has the same external diameter as that of the catheter, and that the machine end of the stop ring has a greater diameter than its patient end to make a smooth transition with the patient end of a dilator slid along the guiding catheter from its machine end and has the same external diameter as that of the patient end of the dilator.

The stop ring preferably tapers outwardly from its patient end to its machine end along substantially the entire length of the stop ring. The length of the stop ring is preferably at least twice the minimum diameter of the stop ring. The length of the stop ring may be approximately three times the minimum diameter of the stop ring. The stop ring may be approximately 6 mm long and taper from a diameter of approximately 1.75 mm at one end to a diameter of approximately 4 mm at its opposite end.

According to another aspect of the present invention there is provided an assembly of a guiding catheter according to the above one aspect of the present invention and a dilator with a patient end abutting the machine end of the stop ring.

The dilator preferably has an S-shape.

According to a further aspect of the present invention there is provided a tracheostomy kit including a tracheostomy tube, a needle for making an initial penetration passage into the trachea, a guidewire for insertion into the passage made by the needle, a guiding catheter according to the above one aspect of the present invention, and a dilator slidable along the guiding catheter and slidable with the guiding catheter through the passage to enlarge the passage sufficiently to receive the tracheostomy tube.

According to yet another aspect of the present invention there is provided a method of inserting a tracheostomy tube including the steps of inserting a guidewire into the trachea through a passage in neck tissue, sliding the patient end of a guiding catheter according to the above one aspect of the present invention along the guidewire and into the trachea without the preliminary step of predilating the passage into the trachea, sliding a dilator with a patient end in contact with the machine end of the stop ring on the guiding catheter together with the guiding catheter along the guidewire into the trachea to enlarge the passage through the tracheal wall, removing the dilator and guiding catheter, inserting a tracheostomy tube on an introducer along the guidewire through the dilated passage and subsequently removing the introducer and guidewire to leave the tube in position.

A guiding catheter, tracheostomy procedure kit and a procedure for inserting a tracheostomy tube will now be described, by way of example, with reference to the accompanying drawings, in which:

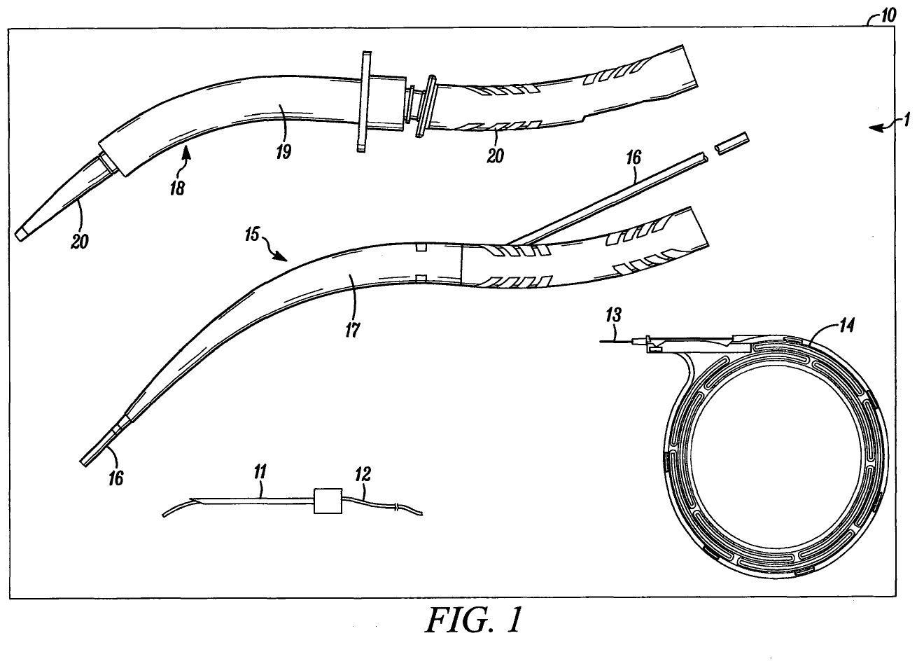

FIG. 1 is a simplified plan view of a tracheostomy procedure kit;

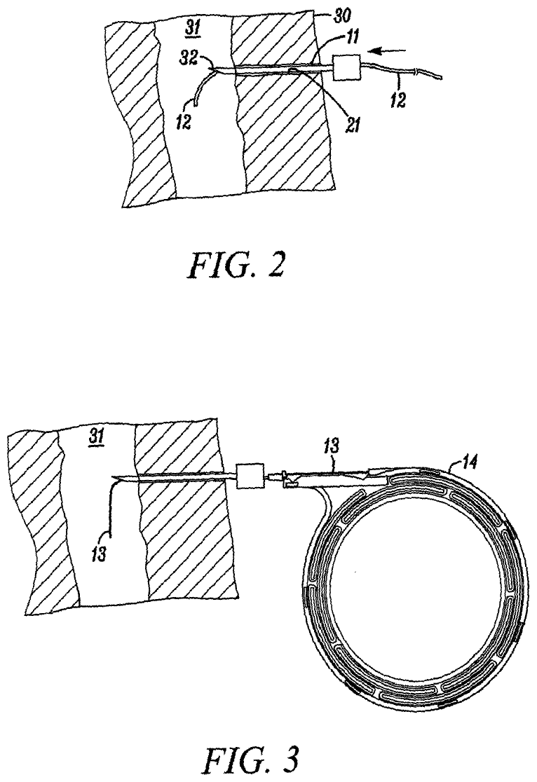

FIGS. 2 to 4 show in simplified form the first three steps in the procedure;

FIG. 5 shows an enlarged elevation view of a part of the guiding catheter and dilator; and

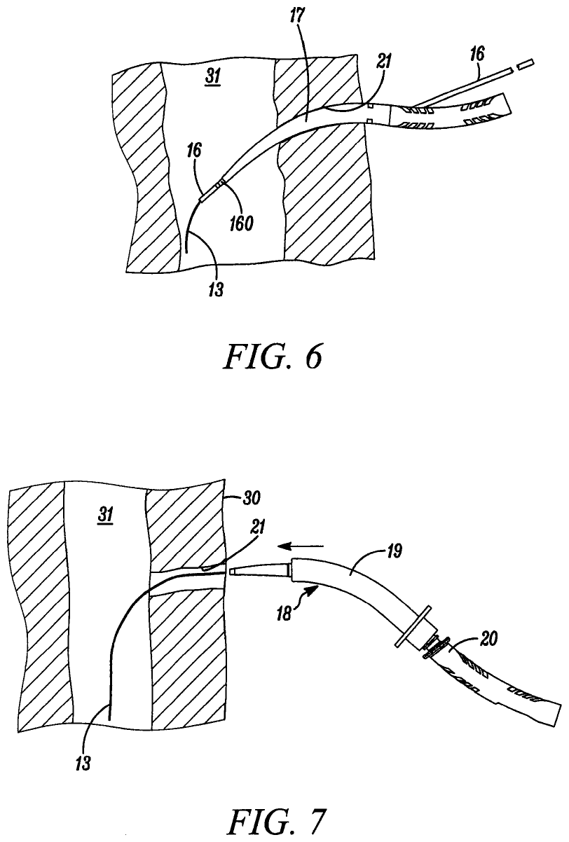

FIGS. 6 and 7 show in simplified form two subsequent steps in the procedure.

With reference first to FIG. 1, the tracheostomy procedure kit 1 is contained in a tray 10 with a peel-off non-woven cover sheet (removed to reveal the contents). The kit 1 contains a needle 11 and cannula 12 extending in the bore of the needle, a guidewire 13 in a holder/dispenser 14, an assembly 15 of a guiding catheter 16 and a single stage dilator 17, and an assembly 18 of a tracheostomy tube 19 and introducer 20. The kit 1 also contains other conventional items such as scalpel, wipes and lubricant and the like, which are not shown.

With reference now to FIG. 2, the first step in the procedure is to make the initial passage 21 through the neck 30 into the trachea 31 by pushing the needle 11 between the second and third tracheal rings so that its tip 32 is located in the trachea. Penetration of the trachea 31 can be confirmed by any of the conventional techniques, such as by connecting a syringe to the hub of the needle and ensuring that air can be freely aspirated. The cannula 12 is then extended from the patient end tip 32 of the needle 11 so that it is directed down the trachea.

The next step, as shown in FIG. 3 is to advance the guidewire 13 through the cannula 12 and needle 11 so that its patient end locates in the trachea 31. The guidewire holder 14 is then removed, leaving the machine end of the guidewire 13 loose extending externally, outside the body.

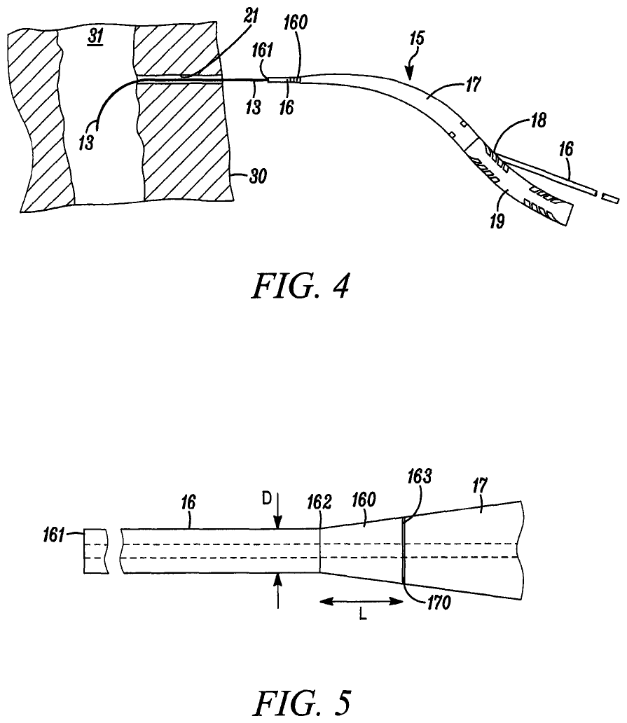

The next step, as shown in FIG. 4, is to thread the assembly 15 of the guiding catheter 16 and dilator 17 along the guidewire 13 up to the skin surface on the neck 30. The guiding catheter 16 is shown in more detail in FIG. 5 and can be seen to have a nylon stop ring 160 overmoulded onto the outer surface of the catheter towards its patient end 161. The purpose of the stop ring 160 is to prevent the guiding catheter 16 being pushed rearwardly along the dilator when the catheter enters the neck tissue. The stop ring 160 is longer than conventional stop rings having a length L that is at least twice and preferably three times the diameter D of the main part of the guiding catheter 16. Typically, the length L of the stop ring 160 is about 6 mm and the diameter D is about 1.75 mm. The patient end 162 of the stop ring 160 has an external diameter equal to that of the guiding catheter 16 itself so that there is a smooth, stepless transition between the catheter and the stop ring 160 where they meet. The stop ring 160 tapers outwardly rearwardly along its entire length at a fairly shallow angle of about 10° so that the machine end 163 of the stop ring has a diameter about twice that of its patient end 162 and of the guiding catheter 16. Typically the diameter of the machine end 163 of the stop ring 160 is about 4.0 mm and is selected to be equal to the external diameter at the forward, patient end tip 170 of the dilator 17. The taper along the stop ring 160 is the same as that along the patient end of the dilator 17 so that there is a smooth continuation in the junction between the stop ring and the dilator. Alternatively, the patient end of the stop ring could have a short, thin-walled extension of constant diameter although this would result in a shallow step between the outer surface of the catheter and of the patient end of the stop ring unless the outer surface of the catheter were formed with a shallow recess to receive the stop ring.

The dilator 17 is tapered and has an S-shape curve along its length with a side opening 18 towards the rear, machine end 19 of the dilator through which the guidewire 13 and guiding catheter 16 emerge. More information about the dilator can be seen in U.S. Pat. No. 8,372,107 but is not essential for an understanding of the present invention.

The guiding catheter 16 and dilator 17 are pushed forwardly along the guidewire 13 so that the patient end 161 of the guiding catheter passes through the passage 21 made through the tracheal tissue into the trachea 31, as shown in FIG. 6. As the guiding catheter 16 passes further into the trachea 31 the patient end 162 of the stop ring 160 starts to pass through the neck tissue. Because the forward, patient end 162 of the stop ring 160 is a smooth continuation of the outside of the guiding catheter 16 it slides through the neck tissue with only a minimal increase in resistance. The shallow, gradual taper along the stop ring 160 ensures that it can pass through the neck tissue smoothly with little increase in resistance. This continues until the forward, patient end tip 170 of the dilator 17 contacts and enters the neck tissue. The matched diameter and taper of the forward end 170 of the dilator 17 with the rear 163 of the stop ring 160 ensures that the junction between the two components does not impede insertion of the assembly. Continued insertion causes a gradual expansion or dilation of the passage 21 through the neck tissue and a gradual but controlled increase in resistance to insertion. The dilator 17 is inserted in the usual way up to an insertion marker at which point the tracheal passage 21 is dilated to the extent necessary for insertion of the tracheostomy tube 19. The assembly 15 of the dilator 17 and guiding catheter 16 is then removed leaving the guidewire 13 in place.

The assembly 18 of the tracheostomy tube 19 and introducer 20 is then slid along the guidewire 13 until the flange of the tube lies against the neck surface. The introducer 20 is then removed and the machine end of the tracheostomy tube is connected to a ventilator or left open to atmosphere as desired.

The shape of the stop ring 160 allows it to act both as a stop ring for the dilator 17 and as a pre-dilator to expand neck tissue sufficiently to enable the dilator to be inserted freely and continuously. This avoids the need for a separate procedure of pre-dilation using separate pre-dilator components. This considerably simplifies the procedure and reduces the different number of components that need to be provided in the procedure kit.

The invention is not limited to use with a single stage dilator but could also be used with a series of dilators of increasing diameters.

1-9. (canceled)

10. A guiding catheter for use with a dilator in a tracheostomy procedure, characterised in that the guiding catheter has an elongated stop ring the length of which is at least twice the minimum diameter of the ring, that the patient end of the stop ring makes a smooth transition with the external surface of the guiding catheter and has the same external diameter as that of the catheter, and that the machine end of the stop ring has a greater diameter than its patient end to make a smooth transition with the patient end of a dilator slid along the guiding catheter from its machine end and has the same external diameter as that of the patient end of the dilator.

11. A guiding catheter according to claim 10, characterised in that the stop ring tapers outwardly from its patient end to its machine end along substantially the entire length of the stop ring.

12. A guiding catheter according to claim 10, characterised in that the length of the stop ring is approximately three times the minimum diameter of the stop ring.

13. A guiding catheter according to claim 10, characterised in that the stop ring is approximately 6 mm long and tapers from a diameter of approximately 1.75 mm at one end to a diameter of approximately 4 mm at its opposite end.

14. An assembly having a guiding catheter and a dilator for use in a tracheostomy procedure, characterised in that the guiding catheter has an elongated stop ring the length of which is at least twice the minimum diameter of the ring, that the patient end of the stop ring makes a smooth transition with the external surface of the guiding catheter and has the same external diameter as that of the catheter, and that the machine end of the stop ring has a greater diameter than its patient end to make a smooth transition with the patient end of the dilator slid along the guiding catheter from its machine end and has the same external diameter as that of the patient end of the dilator.

15. The assembly according to claim 14, characterised in that the dilator has an S-shape.

16. The assembly of claim 14, wherein the guiding catheter and the dilator are parts of a tracheostomy kit that includes a tracheostomy tube, a needle for making an initial penetration passage into the trachea, a guidewire for insertion into the passage made by the needle, and wherein the dilator is slidable along the guiding catheter and slidable with the guiding catheter through the passage to enlarge the passage sufficiently to receive the tracheostomy tube.

17. A method of inserting a tracheostomy tube using a guiding catheter including the steps of inserting a guidewire into the trachea through a passage in neck tissue, sliding the patient end of a guiding catheter having an elongated stop ring the length of which is at least twice the minimum diameter of the ring, that the patient end of the stop ring makes a smooth transition with the external surface of the guiding catheter and has the same external diameter as that of the catheter, and that the machine end of the stop ring has a greater diameter than its patient end to make a smooth transition with the patient end of a dilator slid along the guiding catheter from its machine end and has the same external diameter as that of the patient end of the dilator, the method along the guidewire and into the trachea without the preliminary step of predilating the passage into the trachea, sliding the dilator with its patient end in contact with the machine end of the stop ring on the guiding catheter together with the guiding catheter along the guidewire into the trachea to enlarge the passage through the tracheal wall, removing the dilator and guiding catheter, inserting a tracheostomy tube on an introducer along the guidewire through the dilated passage and subsequently removing the introducer and guidewire to leave the tube in position.
